(19) United States
(12) Patent Application Publication
(10) Pub. No.: US 2020/0211707 A1
(43) Pub. Date: Jul. 2, 2020

(54) SYSTEM AND METHOD FOR PREDICTING TYPES OF PATHOGENS IN PATIENTS WITH SEPTICEMIA

(71) Applicant: NATIONAL CHENG KUNG UNIVERSITY, Tainan City (TW)

(72) Inventors: Po-Lin CHEN, TAINAN CITY (TW); Cheng-Yu TSAI, TAIPEI CITY (TW); Bing-Ze LU, CHANGHUA COUNTY (TW); Yu-Chen SHU, TAINAN CITY (TW); Nai-Ying KO, KAOHSIUNG CITY (TW); Chun-Yin YEH, TAINAN CITY (TW); Wen-Chien KO, TAINAN CITY (TW); Kun-Ta CHUANG, TAINAN CITY (TW); Hung-Yu KAO, TAINAN CITY (TW)

(21) Appl. No.: 16/664,938

(22) Filed: Oct. 27, 2019

Related U.S. Application Data
(60) Provisional application No. 62/785,699, filed on Dec. 28, 2018.

(30) Foreign Application Priority Data
Aug. 21, 2019 (TW) 108129894

(51) Int. Cl. G16H 50/20; A61B 5/0205; A61B 5/11

(52) U.S. Cl. CPC G16H 50/20; G06N 20/20; A61B 5/11; A61B 5/02055

(57) ABSTRACT

A system for predicting types of pathogens in patients with septicemia is provided. The system includes at least one sensor and a processor. The sensor is used to sense current physiological data including at least one of body temperature, blood pressure, and pulse. The processor is configured to calculate at least one feature value according to the current physiological data, and input the feature value into a machine learning model to determine one of categories including at least two of uninfected, fungal infected, contaminated bacteria infected, Gram-negative infected, and Gram-positive infected.

### RELATED APPLICATIONS

This application claims priority to U.S. Provisional Application Ser. No. 62/785,699 filed Dec. 28, 2018, and Taiwan Application Serial Number 108129894, filed Aug. 21, 2019, the disclosures of which are incorporated herein by reference in their entireties.

### BACKGROUND

### Field of Invention

The present invention relates to a method and a system for predicting types of pathogens in patents with septicemia before the culture result of the pathogenic bacteria.

### Description of Related Art

Sepsis is the major death cause of the hospitalized patents. Administration of effective antibiotics can decrease the mortality rate of the patents with septicemia. However, the method of accurately identifying the type of pathogens before the culture results available is still lacking. Physicians usually give empiric antibiotics based on individual judgement without solid supporting evidences. It is concerned by people in the field about how to determine whether a patient is infected or infected by what type of pathogen before the culture result of the pathogen is available.

### SUMMARY

Embodiments of the present disclosure provide a system for predicting types of pathogens in patients with septicemia. The system includes at least one sensor and a processor. The sensor is configured to sense current physiological data, wherein a type of the current physiological data includes at least one of body temperature, blood pressure, and pulse. The processor is configured to calculate at least one feature value according to the current physiological data, and input the feature value into a machine learning model to determine one of categories including at least two of uninfected, fungal infected, contaminated bacteria infected, Gram-negative infected, and Gram-positive infected.

In some embodiments, the processor is further configured to perform steps of: obtaining healthy physiological data that changes over time; calculating a mean of the healthy physiological data as a healthy mean; calculating a variance of the healthy physiological data as a healthy variance; calculating a variance of the current physiological data as a current variance; calculating a variance of the current physiological data respect to the healthy mean as a reference variance; dividing the reference variance by the healthy variance as a first feature value; and dividing the current variance by the healthy variance as a second feature value.

In some embodiments, the reference variance is calculated according to the following equation (1) where X_{current} is a value of samples of the current physiological data, μₕₑₐₗₜₕ is the healthy mean, and # current is a number of the samples of the current physiological data.

$$\frac{{Σ ( {X}_{current} - {μ}_{health} )}^{2}}{\# current} \qquad ( 1 )$$

In some embodiments, the at least one sensor includes a gravity sensor, and the processor is configured to determine if a user is stationary according to a signal sensed by the gravity sensor, and obtain the current physiological data only when the user is stationary.

In some embodiments, the machine learning model is a random forest algorithm.

In some embodiments, the processor is further configured to generate an image for each type of the current physiological data according to a following equation (2).

p_{i,j}=(X_{current,i}−μ_{current})×(X_{health,j}−μₕₑₐₗₜₕ) (2)

p_{i,j} is a pixel at i^{th} column and j^{th} row of the image. X_{current,i} is a i^{th} value of the current physiological data. and X_{health,j} is j^{th} value of the healthy physiological data. i and j are positive integers. The processor is further configured to input the image into a convolutional neural network to determine one of the categories.

From another aspect, a method for predicting types of pathogens in patients with septicemia that is performed on a processor is provided. The method includes: sensing, by at least one sensor, current physiological data in which a type of the current physiological data includes at least one of body temperature, blood pressure, and pulse; and calculating, by a processor, at least one feature value according to the current physiological data, and inputting the feature value into a machine learning model to determine one of categories including at least two of uninfected, fungal infected, contaminated bacteria infected, Gram-negative infected, and Gram-positive infected.

### BRIEF DESCRIPTION OF THE DRAWINGS

The invention can be more fully understood by reading the following detailed description of the embodiment, with reference made to the accompanying drawings as follows.

FIG. 1 is a schematic diagram illustrating of a system for predicting types of pathogens in accordance with an embodiment.

FIG. 2 is a flow chart for classifying types of pathogens in accordance with an embodiment.

FIG. 3 is a flow chart of a method for predicting types of pathogens in accordance with an embodiment.

### DETAILED DESCRIPTION

Specific embodiments of the present invention are further described in detail below with reference to the accompanying drawings, however, the embodiments described are not intended to limit the present invention and it is not intended for the description of operation to limit the order of implementation. Moreover, any device with equivalent functions that is produced from a structure formed by a recombination of elements shall fall within the scope of the present invention. Additionally, the drawings are only illustrative and are not drawn to actual size.

The using of “first”, “second”, “third”, etc. in the specification should be understood for identifying units or data described by the same terminology, but are not referred to particular order or sequence.

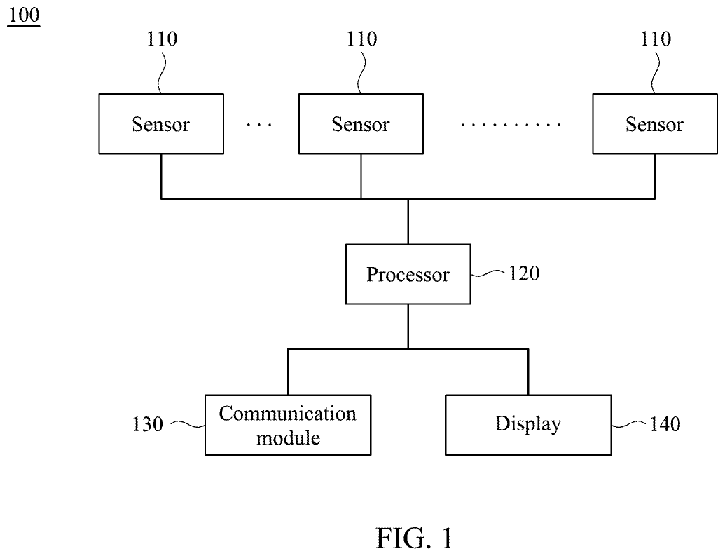

FIG. 1 is a schematic diagram illustrating of a system for predicting types of pathogens in accordance with an embodiment. Referring to FIG. 1, a system 100 includes multiple sensors 110, a processor 120, a communication module 130, and a display 140. The sensors 110 are configured to sense physiological data. The type of the sensed physiological data includes body temperature, blood pressure (including diastolic and systolic pressures), pulse, heat rate, etc. People in the field should be able to choose appropriate sensors to sense the corresponding physiological data. For example, an infrared thermometer may be used to sense the body temperature, and so on. The processor 120 may be a central processing unit, a microprocessor, a microcontroller, a digital signal processor, an application-specific integrated circuit, etc. The communication module 130 may be a wire or wireless communication circuit for communicating with other devices. For example, the communication module 130 could be a circuit with functions of universal serial bus (USB), the Internet, local area networks (LANs), wide area networks (WANs), cellular telephone networks, near field communications (NFC), infrared (IR), Bluetooth, or WiFi. The display 140 may be liquid crystal display, organic light emitting diode (OLED) display, or other suitable displays. In the embodiment, the sensors 110 sense current physiological data, and the processor 120 calculates feature values according to the current physiological data, and inputs the feature values into a machine learning model to determine one of categories including virus infected, uninfected, fungal infected, contaminated bacteria infected, Gram-negative infected, Gram-positive infected, etc. In some embodiments, the system 100 is implemented as a wristband carried on patient's hand. In other embodiments, the system 100 may be implemented as any form of computer or mobile devices, which are not limited in the invention. In other embodiments, the system 100 may include other suitable components or circuits, and the communication module 130 and the display 140 may be omitted.

How the types of infection are determined is described herein. First, the physiological data of body temperature, blood pressure, pulse, and heat rate are signals that change overtime. The processor 120 obtains the physiological data in a period (e.g. few seconds, but the length of the period is not limited) through the sensors 110. For example, if the sampling frequency is 60 Hz, then five seconds of the physiological data include 60×5=300 values. The sampling frequency is not limited in the invention. The physiological data obtained by the sensors 110 are referred to current physiological data.

In addition, the processor 120 obtains physiological data of body temperature, blood pressure, pulse, and heat rate corresponding to a healthy state from a database (not shown). The obtained data is also referred to healthy physiological data. The healthy physiological data is measured from the people who are healthy (e.g. uninfected). The healthy physiological data also changes overtime, but the length and the sampling frequency of the healthy physiological data are not limited in the invention. In other words, the length of the healthy physiological data may be different from that of the current physiological data.

The processor 120 calculates two features values for each type of the physiological data (i.e. body temperature, blood pressure, pulse, or heat rate). Herein, a value of a sample included in the healthy physiological data is written as Xₕₑₐₗₜₕ. # health is the number of the values Xₕₑₐₗₜₕ. # health is also referred to the length of the healthy physiological data. A value of a sample included in the current physiological data is written as X_{current}. # current is the number of the values X_{current}. # current is also referred to the length of the current physiological data or the number of the samples of the current physiological data. The processor 120 calculates the man of the healthy physiological data as a healthy mean which is written as μₕₑₐₗₜₕ in the following equations. The mean of the current physiological data is written as μ_{current}. In addition, the variance of the healthy physiological data is calculated based on the following equation (1) as a healthy variance σₕₑₐₗₜₕ. The variance of the current physiological data is calculated based on the following equation (2) as a current variance σ_{sick-sick}. The variance of the current physiological data with respect to the healthy mean is calculated based on the following equation (3) as a reference variance σ_{current-health}.

$$\begin{matrix}{σ}_{health} = \frac{{Σ ( {X}_{health} - {μ}_{health} )}^{2}}{\# health} & ( 1 ) \\ {σ}_{sick \text{-} sick} = \frac{{Σ ( {X}_{current} - {μ}_{current} )}^{2}}{\# current} & ( 2 ) \\ {σ}_{current \text{-} health} = \frac{{Σ ( {X}_{current} - {μ}_{health} )}^{2}}{\# current} & ( 3 )\end{matrix}$$

A first feature value f1 is obtained by dividing the reference variance by the healthy variance that is written in the following equation (4). A second feature value f2 is obtained by dividing the current variance by the healthy variance that is written in the equation (5).

$$\begin{matrix}f 1 = \frac{{σ}_{current \text{-} health}}{{σ}_{health}} & ( 4 ) \\ f 2 = \frac{{σ}_{sick \text{-} sick}}{{σ}_{health}} & ( 5 )\end{matrix}$$

There are four types of physiological data (i.e. body temperature, blood pressure, pulse, and heat rate) in the embodiment, and therefore total of eight features values (including four of first feature values f1 and four of second feature values f2) are calculated. Alternatively, the blood pressure includes diastolic blood pressure that corresponds to two feature values f1 and f2 and systolic blood pressure that corresponds to two feature values f1 and f2, and thus total of ten feature values are calculated. All the calculated first feature values f1 and second feature values f2 constitute a feature vector which is inputted into a machine learning model such as a random forest algorithm, a support vector machine, a neural network, and so on that is not limited in the invention. The machine learning model is trained to determine if the patients are infected and determine the types of the pathogens. In some embodiments, the categories outputted by the machine learning model include at least two of virus infected, uninfected, fungal infected, contaminated bacteria infected, Gram-negative infected, and Gram-positive infected. Herein, “contaminated bacteria infected” means that the pathogen in the patient is caused by some sources of pollution instead of sepsis.

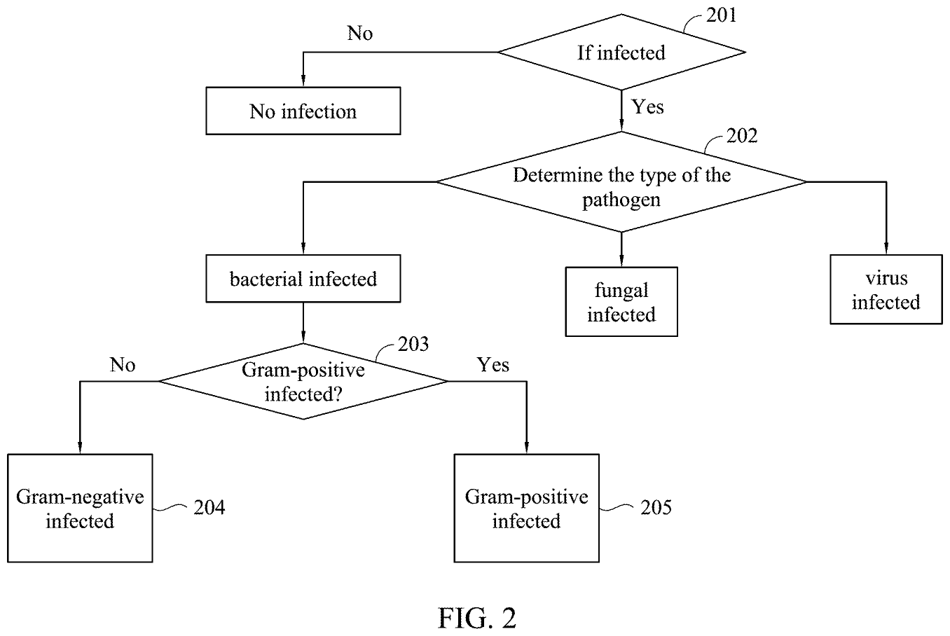

Referring to FIG. 2, in some embodiments, the step 201 is performed first to determine if the patient is infected. If the result of the step 201 is no, it means there is no infection. If the result of the step 201 is affirmative, the step 202 is then performed to determine the type of the pathogen as bacterial infected, fungal infected, or virus infected. If the type of bacterial infected is determined, then in the step 203, it is determined if the patient is Gram-positive infected. It is determined to be Gram-negative infected (step 204) or Gram-positive infected (step 205) according to the result of step 203. In some embodiments, three classifiers are trained that correspond to the steps 201 to 203 respectively. In other embodiments, only one classifier is trained to output categories of uninfected, fungal infected, virus infected, Gram-negative infected, and Gram-positive infected. Note that the flow of FIG. 2 is merely an example, and other steps may be added into FIG. 2 or some steps may be removed from FIG. 2. For example, the category of contaminated bacteria infected is also determined in the step 202 in some embodiments.

Among the aforementioned physiological data, body temperature is important for determining if the patient is infected. However, the patient may get up and move, and thus affecting the value of the body temperature. In some embodiments, the sensors 110 of FIG. 1 include a gravity sensor such as an acceleration sensor. It is determined if the user is stationary according to the signals of the gravity sensor. For example, the user is determined to be stationary when the accelerations at all directions are less than a threshold. In addition, the current physiological data is acquired only when the user is stationary. That is, when the user is not stationary, the processor 120 would ignore the physiological data sensed by the sensor 110. In this way, it is possible to avoid obtaining an inappropriate body temperature when the user moves or performs other actions, and thus the determination of infection is improved.

Note that the feature values f1 and f2 are merely a portion of the feature vector which may include other information such as user's age, gender, medical history that would be digitized as part of the feature vector. Alternatively, the signals sensed by the sensors 110 may be used to calculate other feature values to constitute the feature vector, which is not limited in the invention.

In some embodiments, the system 100 is a wearable device carried by the patient who can go anywhere. The system 100 can determine whether the patient is infected from time to time or periodically, and can also transmit the collected physiological data or the classification result to a server or the doctor's mobile phone through the communication module 130. This allows the hospital or doctor to notify the patient to seek immediate medical attention for effective medical treatment.

In some embodiments, the physiological data is transformed into images which are inputted into a convolutional neural network for classification. For example, for each type of physiological data, an image is generated according to co-variance between the current physiological data and the healthy physiological data. To be specific, the pixel at i^{th} column and j^{th} row of the image is written as p_{i,j} calculated as the following equation (6). X_{current,i} is the i^{th} value of the current physiological data, and X_{health,j} is the j^{th} value of the healthy physiological data where i and j are positive integers.

p_{i,j}=(X_{current,i}−μ_{current})×(X_{health,j}−μₕₑₐₗₜₕ) (6)

Each type of the physiological data can be used to generate one image, and therefore total of four images are generated. The four images are combined as a two-dimensional image with four channels. This two-dimensional image is inputted to a convolutional neural network to perform the classification. From another aspect, the pixel p_{i,j} may be referred to a feature value.

In some embodiments, an image is generated based on the following equation (7).

p_{i,j}=(xᵢ−xⱼ)² (7)

xᵢ is the i^{th} value of the current physiological data or the healthy physiological data. Note that the equation (7) can be applied to both of the current physiological data and the healthy physiological data, and hence two images can be generated for each type of the physiological data. Accordingly, total of eight images are generated and combined as a two-dimensional image with eight channels. The two-dimensional image is inputted into a convolutional neural network to perform the classification.

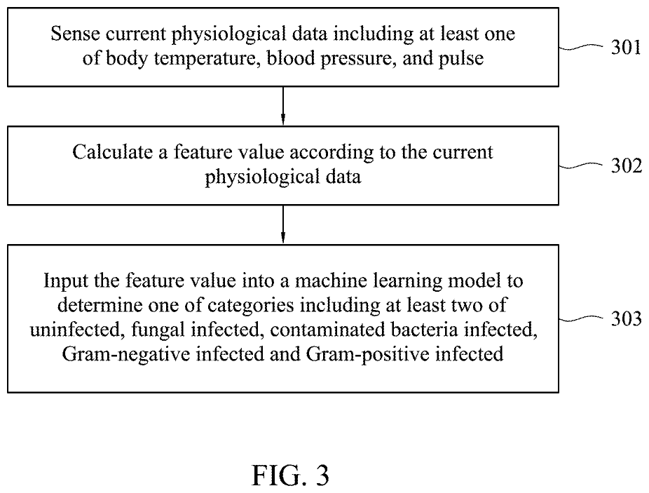

FIG. 3 is a flow chart of a method for predicting types of pathogens in accordance with an embodiment. Referring to FIG. 3, in step 301, current physiological data is sensed, in which a type of the current physiological data includes at least one of body temperature, blood pressure, and pulse. In step 302, a feature value is calculated according to the current physiological data. In step 303, the feature value is inputted into a machine learning model to determine one of categories including at least two of uninfected, fungal infected, contaminated bacteria infected, Gram-negative infected and Gram-positive infected. However, all the steps in FIG. 3 have been described in detail above, and therefore the description they will not be repeated. Note that the steps in FIG. 3 can be implemented as program codes or circuits, and the disclosure is not limited thereto. In addition, the method in FIG. 3 can be performed with the aforementioned embodiments, or can be performed independently. In other words, other steps may be inserted between the steps of the FIG. 3.

In the system and method described above, whether the patient is infected and the types of the pathogen can be predicted before the blood culture result is released. In addition, the clinician can refer to the predicted result to open a suitable antibiotic to treat the sepsis patient, resulting in higher survival rate of sepsis patients. Moreover, the prediction method is non-invasive, and no additional blood test is needed.

Although the present invention has been described in considerable detail with reference to certain embodiments thereof, other embodiments are possible. Therefore, the spirit and scope of the appended claims should not be limited to the description of the embodiments contained herein. It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. A system for predicting types of pathogens in patients with septicemia, wherein the system comprises:
at least one sensor, configured to sense current physiological data, wherein a type of the current physiological data includes at least one of body temperature, blood pressure, and pulse; and
a processor, configured to calculate at least one feature value according to the current physiological data, and input the at least one feature value into a machine learning model to determine one of categories comprising at least two of uninfected, fungal infected, contaminated bacteria infected, Gram-negative infected, and Gram-positive infected.

2. The system of claim 1, wherein the processor is further configured to perform steps of:
obtaining healthy physiological data that changes over time;
calculating a mean of the healthy physiological data as a healthy mean;
calculating a variance of the healthy physiological data as a healthy variance;
calculating a variance of the current physiological data as a current variance;
calculating a variance of the current physiological data respect to the healthy mean as a reference variance;
dividing the reference variance by the healthy variance as a first feature value; and
dividing the current variance by the healthy variance as a second feature value.

3. The system of claim 2, wherein the reference variance is calculated according to a following equation (1):
$$\frac{{Σ ( {X}_{current} - {μ}_{health} )}^{2}}{\# current} \qquad ( 1 )$$
wherein X_{current} is a value of one of a plurality of samples of the current physiological data, μₕₑₐₗₜₕ is the healthy mean, and # current is a number of the samples of the current physiological data.

4. The system of claim 1, wherein the at least one sensor comprises a gravity sensor, and the processor is configured to determine if a user is stationary according to signals sensed by the gravity sensor, and obtain the current physiological data only when the user is stationary.

5. The system of claim 1, wherein the machine learning model is a random forest algorithm.

6. The system of claim 1, wherein the processor is further configured to perform steps of:
generating an image for each type of the current physiological data according to a following equation (1):
p_{i,j}=(X_{current,i}−μ_{current})×(X_{health,j}−μₕₑₐₗₜₕ) (1)
wherein p_{i,j} is a pixel at i^{th} column and j^{th} row of the image, X_{current,i} is a i^{th} value of the current physiological data, and X_{health,j} is a j^{th} value of the healthy physiological data where i and j are positive integers; and
inputting the image into a convolutional neural network to determine one of the categories.

7. A method for predicting types of pathogens in patients with septicemia that is performed on a processor, wherein the method comprises:
sensing, by at least one sensor, current physiological data, wherein a type of the current physiological data includes at least one of body temperature, blood pressure, and pulse; and
calculating, by a processor, at least one feature value according to the current physiological data, and inputting the at least one feature value into a machine learning model to determine one of categories comprising at least two of uninfected, fungal infected, contaminated bacteria infected, Gram-negative infected, and Gram-positive infected.

8. The method of claim 7, wherein the step of calculating the at least one feature value according to the current physiological data comprises:
obtaining healthy physiological data that changes over time;
calculating a mean of the healthy physiological data as a healthy mean;
calculating a variance of the healthy physiological data as a healthy variance;
calculating a variance of the current physiological data as a current variance;
calculating a variance of the current physiological data respect to the healthy mean as a reference variance;
dividing the reference variance by the healthy variance as a first feature value; and
dividing the current variance by the healthy variance as a second feature value.

9. The method of claim 8, wherein the reference variance is calculated according to a following equation (1):
$$\frac{{Σ ( {X}_{current} - {μ}_{health} )}^{2}}{\# current} \qquad ( 1 )$$
wherein X_{current} is a value of one of a plurality of samples of the current physiological data, μₕₑₐₗₜₕ is the healthy mean, and # current is a number of the samples of the current physiological data.

10. The method of claim 7, further comprises:
determining if a user is stationary according to signals sensed by a gravity sensor, and obtaining the current physiological data only when the user is stationary.

11. The method of claim 7, wherein the machine learning model is a random forest algorithm.
